(19) United States
(12) Patent Application Publication
(10) Pub. No.: US 2022/0261287 A1
(43) Pub. Date: Aug. 18, 2022

(54) METHOD AND APPARATUS FOR IMPROVING PROCESSOR RESOURCE UTILIZATION DURING PROGRAM EXECUTION

(71) Applicant: NVIDIA Corporation, Santa Clara, CA (US)

(72) Inventors: Shekhar Dwivedi, Santa Clara, CA (US); Andreas Heumann, Polling (DE)

(21) Appl. No.: 17/174,951

(22) Filed: Feb. 12, 2021

(51) Int. Cl. G06F 9/50; G16H 30/40; G06N 3/04; G06F 9/38; G06T 1/20

(52) U.S. Cl. CPC G06F 9/5027; G16H 30/40; G06T 2210/41; G06F 9/3836; G06T 1/20; G06N 3/04

(57) ABSTRACT

Systems and methods for improving the degree to which programs utilize processor resources during execution. A number of different versions of a program are received, as is a set of performance metrics describing desired performance of the program versions. The programs are then analyzed to determine the amount of processor resources used on a particular processor when the programs are executed to meet the performance metrics. At runtime, a program version that meets its performance metrics without exceeding the available processor resources is selected for execution by the processor. Program versions may be versions written to utilize processors in differing manner, such as by adjusting the numerical precision at which operations are performed or stored. If no program version meets its performance metrics without exceeding the available processor resources, the performance metrics may be reduced and program selection may be based on these reduced performance metrics.

### BACKGROUND

Embodiments of the disclosure relate generally to electronic computing systems. More specifically, embodiments of the disclosure relate to improving runtime resource utilization of electronic computing systems.

### SUMMARY

Advances in computer processor technology have improved almost every aspect of processor performance. Various processor types, such as central processing units (CPUs) and graphics processing units (GPUs), continue to improve in architecture, processing speed, memory capacity and usage, and many other metrics. However, software applications—even those developed on or for the latest hardware platforms available at the time of the development—may not always be able to take full advantage of the improved performance capabilities of newer processors, and thus often do not run optimally.

Accordingly, systems and methods are described herein for improving processor resource utilization of programs or applications during their execution. In exemplary embodiments of the disclosure, the processor resource utilizations of a number of variations of a particular program or process are determined. When the program is to be run on a particular processor, the available computation resources of that processor are compared to the resource utilizations of the various versions of the program. The version that most optimally utilizes the processor's resources is the version selected for execution.

In some embodiments of the disclosure, a table or other compilation of the computational resources consumed by execution of each program variation is generated, and compared to the available computational resources of the particular processor which is to execute the program. In particular, one or more performance metrics are received or otherwise selected. At or prior to runtime, the table may be analyzed to determine which, if any, program version satisfies the performance metrics on the particular processor without exceeding the computational resources of the processor. For example, performance metrics may include criteria such as an end to end runtime or the like (e.g., the program must generate results within some specified time period). Computational resources may include, e.g., the floating point operations per second (FLOPS) that the processor is capable of executing, the amount of available processor memory, or the like. Thus, for example, the above mentioned table may be analyzed to determine which, if any, program may be run to completion within the specified time, without exceeding the processor's available FLOPS or memory (whether onboard or associated). A program version which satisfies (or the program version which best satisfies) the performance metrics without exceeding the processor's resources may be the version selected for execution.

If none of the program versions can meet the performance metrics for a given processor without exceeding the processor's resources, a number of actions may be taken. The performance metrics may be relaxed, for one example. That is, the program version which satisfies the reduced performance metrics without exceeding the processor's resources may be selected for execution. If no program version can satisfy the reduced performance metrics without exceeding the processor's resources, the program version which most closely satisfies the reduced performance metrics without exceeding the processor's resources may be selected for execution.

The performance metrics may be any metrics by which program or application performance may be measured. For example, performance metrics may include any one or more of end-to-end runtime, or any minimum rate at which information may be generated. For example, for some medical imaging applications, the minimum rate may be any minimum rate at which medical images may be generated to support a particular objective, such as real time viewing. Accordingly, reduced performance metrics may be any reduced or lowered version of such metrics, e.g., a longer end-to-end runtime, a lowered minimum rate at which information may be generated, or the like.

Computational resources may be any resources of any processor. Such resources may include, for example, memory resources such as memory available for use in execution of any program. Resources may also include processor operations resources such as processor FLOPS, the total number of operations performed in execution of the program in question, or the like. thus, any processor resources may be considered in determining whether the program versions can meet the performance metrics for the processor without exceeding the processor's resources. For instance, it may be separately determined whether any program version can meet the performance metrics without exceeding either the processor's memory resources or operations resources. If no program version can meet the performance metrics without exceeding the processor's memory resources, i.e. if program execution is memory bound, memory-related performance metrics may be reduced, and/or program versions may be selected which most closely meet these reduced memory-related metrics. Similarly, of no program version can meet the performance metrics without exceeding the processor's operations resources, i.e. if program execution is compute-bound, operations-related performance metrics may be reduced, and/or program versions may be selected which most closely meet these reduced operations-related metrics.

It is noted that the programs in question may be any computer-executable program. As one example, programs may be medical image construction or reconstruction programs, or any other programs for generating medical images. However, any programs for use in any field are contemplated by various embodiments of the disclosure.

It is also noted that embodiments of the disclosure may be employed in connection with any type of processor. For example, embodiments may be used to improve resource utilization of GPUs, CPUs, or any other type of processor capable of executing one or more programs.

### BRIEF DESCRIPTION OF THE FIGURES

The above and other objects and advantages of the disclosure will be apparent upon consideration of the following detailed description, taken in conjunction with the accompanying drawings, in which like reference characters refer to like parts throughout, and in which:

FIGS. 1 and 2 are block diagrams conceptually illustrating operation of a system for improving processor resource utilization according to embodiments of the disclosure;

FIG. 3 illustrates tabulated program versions and their use in improving processor resource utilization according to embodiments of the disclosure;

FIG. 4 is a generalized embodiment of an illustrative processor constructed for use according to embodiments of the disclosure;

FIG. 5 is a generalized embodiment of a further processor constructed for use according to embodiments of the disclosure;

FIG. 6 is a flowchart illustrating process steps for improving processor resource utilization according to embodiments of the disclosure; and

FIG. 7 is a block diagram conceptually illustrating operation of a system for improving processor resource utilization according to further embodiments of the disclosure.

### DETAILED DESCRIPTION

In one embodiment, the disclosure relates to systems and methods for improving the degree to which programs utilize processor resources during runtime execution. A number of different versions of a program are received, as is a set of performance metrics describing desired performance of the program versions. The programs are then analyzed to determine the amount of processor resources used on a particular processor when the programs are executed to meet the performance metrics. At runtime, a program version that meets the provided performance metrics given the available processor resources is selected for execution by the processor. Program versions may be versions written to utilize processors in differing manner, such as by adjusting the numerical precision at which operations are performed or stored. If no program version meets the provided performance metrics without exceeding the available processor resources, the performance metrics may be reduced, and program selection may be based on these reduced performance metrics.

In this manner, different program versions, e.g., differing instruction sets for carrying out the same computational process or processes in varying manner, may be packaged together with instruction sets for carrying out the program version selection processes described herein, to provide a package that may be loaded on any computing device for execution by any processor. The package may automatically select the optimal program version for execution on that processor. Thus, embodiments of the disclosure provide programs that automatically adapt to best utilize whatever processor they are run on, improving processor utilization and efficiency.

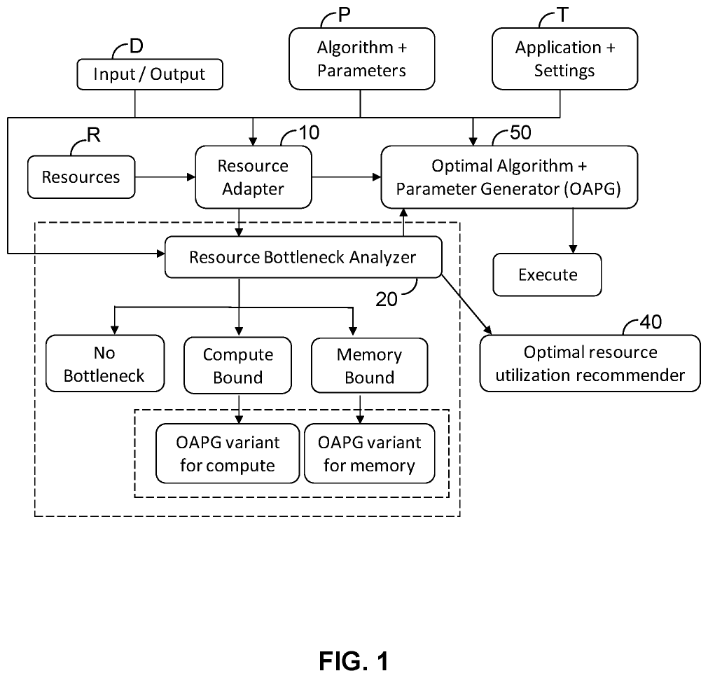

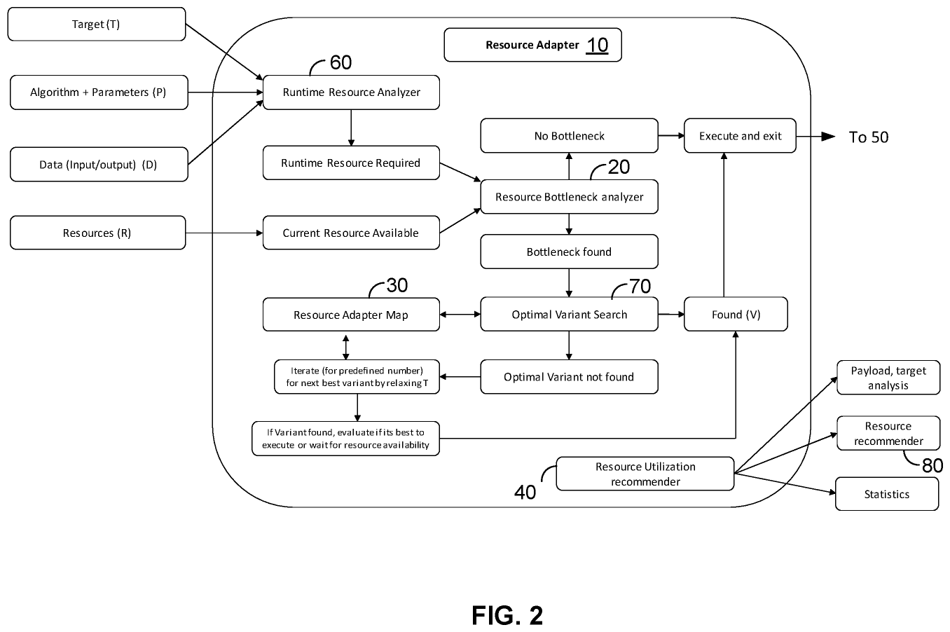

FIGS. 1 and 2 are block diagrams conceptually illustrating operation of a system for improving processor resource utilization according to embodiments of the disclosure. In embodiments of the disclosure, the dashed line of FIG. 1 may indicate logical blocks of resource adapter 10, as shown in FIG. 2. However, embodiments of the disclosure contemplate any arrangement of logical blocks suitable for carrying out the processes described below, and accordingly logical blocks may be arranged in various ways. As one example, block 40 is shown outside of the dashed line of FIG. 1, but is shown within resource adapter 10 in FIG. 2. Various blocks such as block 40 may thus be considered to be within or outside of any other block as desired.

Here, a number of software modules may analyze the performance of various program versions on a particular processor, to select a version best suited for execution on that processor. The software modules include a resource adapter 10, a resource bottleneck analyzer 20, a resource analyzer map 30, a resource utilization recommender 40, and an optimal algorithm and parameter generator (OAPG) 50. The resource adapter 10 has a runtime resource analyzer 60 that receives as inputs a target T which is the performance metric(s) that the program is to meet, as well as a number of algorithms and their parameters P, which are the various program versions and the parameters P that they employ. The program versions comprise different versions of the same program written to utilize processor resources differently, and comprise one default version, or a version whose parameters are selected so that program performance is not compromised, and alternate versions or versions whose parameters are selected to utilize fewer computational resources, or to utilize computational resources in different ways. For example, the default program version may be written to pass data to the processor at a higher numerical precision, e.g., double precision, for accuracy of results, while alternate versions may be written to pass data to the processor at lower numerical precisions, e.g., single precision, to improve memory bandwidth. Similarly, the default version may be written so that the processor operates on data of higher numerical precision to improve processor performance, while alternate versions may be written so that the processor operates on data at lower precision levels. Any variations of any parameters for alternate versions are contemplated. Parameters P may include, for example, the numerical precision level, type of algorithm(s) employed, or any other parameters utilized to influence or control processor compute and/or memory used. The runtime resource analyzer 60 of resource adapter 10 also takes as input the data payload of the programs D or amount of data input to/output by the program versions. From these inputs, runtime resource analyzer 60 determines the runtime resources required by the various program versions, e.g., the maximum amount of processor memory used and the maximum FLOPS performed. Runtime resource determination may be accomplished in any manner, such as via simulation, theoretical calculation, measurement of execution of the program versions on a processor, or the like. Targets T may be any performance metric measuring any desired aspect of program performance, such as end-to-end runtime, any rate of generation of output (e.g., images generated per unit of time), or the like.

The runtime resources consumed by each program are then stored as tables of resource analyzer map 30 and input to resource bottleneck analyzer 20, along with the maximum available resources R (e.g., FLOPS available, processor memory, total operations available, available GPUs, available virtual GPUs (vGPUs), etc.) of the processor on which the program is to be executed. Resource bottleneck analyzer 20 compares the required runtime resources of each program version (output from runtime resource analyzer 10) to current resources available, as determined from the input available resources R of the processor in question. For example, available memory and compute resources may be entered as parameters R, or available computing resources may be determined from other entries such as the number and types of available GPUs or vGPUs. Currently available resources may be determined from parameters R in any manner, such as by simply reading input memory or processor capability values, or by converting input parameters R to currently available resources. Conversion may be performed in any manner, such as by determining the available processor resources corresponding to a specified input number and specification of GPUs or vGPUs. Additionally, vGPU resources may be split or selected in any manner. That is, a proportion of maximum available vGPU resources may be allocated for execution of program versions, where this split may be determined in any manner. In some embodiments of the disclosure, a predetermined amount or percentage of available vGPU resources may be selected, where this predetermined amount may vary according to the specific application (e.g., with application types known to require greater resources being assigned greater amounts of processor resources), according to estimates of computer overhead that are dynamically estimated at runtime, or in any other manner. Alternatively, vGPU resources may be split according to program payload D, with larger payloads corresponding to greater vGPU resources allocated. In some embodiments of the disclosure, determination of available resources may include creation of vGPUs to supply sufficient compute power. In this case, input resources R may include a maximum number of vGPUs or the like, and bottleneck determination may include spawning of additional vGPUs up to this maximum number. Conversely, bottleneck determination may also reduce the number of vGPUs to prevent fragmentation, if fewer compute resources are needed.

If the default program version uses fewer runtime resources than the processor's currently available resources, i.e., if every runtime resource requirement of the default program version is less than the corresponding available resource of the processor in question, then the resource bottleneck analyzer 20 determines that no bottleneck exists (i.e., the No Bottleneck block of FIGS. 1 and 2). The resource bottleneck analyzer 20 may then pass the default program version to the OAPG 50, which initiates execution of the default program version on the processor in question.

If, however, the default program version uses more runtime resources than the processor's currently available resources, the resource bottleneck analyzer 20 determines that a bottleneck exists (i.e., the Bottleneck Found block of FIG. 2). The resource bottleneck analyzer 20 then determines whether the default program is compute bound (e.g., Compute Bound block of FIG. 1) or memory bound (e.g., Memory Bound block of FIG. 1), i.e., whether its resource requirements exceed the processor compute capability or its available memory, as specified by the input resources R. If the default program is compute-bound, the resource bottleneck analyzer 20 compares the required runtime resources of each alternate program version to the available processor resources to determine whether any alternate version falls within the processor's compute capability. That is, as the alternate program versions are written to utilize fewer processor resources (possibly at reduced performance, accuracy, or the like) or to utilize processor resources differently, an optimal variant search engine 70 determines whether any alternate version meets the processor resource requirements while still providing acceptable performance (i.e., exceeding the input target performance, even if not to the same degree as the default program version). If an alternate version falls within the processor resource requirements while still meeting the target performance level, i.e., if an alternate version is not compute-bound, the optimal variant search engine 70 passes that program version to OAPG 50 for execution. If more than one alternate version satisfies both the processor resource requirements and the target performance metrics, the optimal variant search engine 70 selects one of these alternate versions for execution. Selection may be performed in any manner, such as by selecting the alternate version that most exceeds the target performance metrics or offers the highest performance, the version that utilizes the least processor resources, or the like. Additionally, resource utilization recommender 40 may select the alternate program version that best meets some specified criteria, where the criteria may be any input criteria, such as known payload and target analysis methods, known resource selection or recommendation methods, or based on any known statistical or other resource measure.

If the default program is memory bound, the optimal variant search engine 70 compares the memory usage of each alternate program version to the available processor memory. If an alternate version utilizes less processor memory than that available while still meeting or exceeding the target performance metrics, the optimal variant search engine 70 passes that program version to OAPG 50 for execution. If more than one alternate version uses less than the maximum available processor memory while still meeting the target performance metrics, the optimal variant search engine 70 selects one of these alternate versions for execution. Selection may be performed in any manner, such as by selecting the alternate version that most exceeds the target performance metrics or offers the highest performance, the version that utilizes the least processor resources, or the like. Additionally, resource utilization recommender 40 may select the alternate program version that best meets some specified criteria, where the criteria may be any input criteria, such as known payload and target analysis methods, known resource selection or recommendation methods, or based on any known statistical or other resource measure.

If every alternate program version is either compute bound or memory bound, the optimal variant search engine 70 may reduce the target performance metrics to determine whether some program version meets these reduced metrics without exceeding the processor resources. The reduced target performance metrics may be input as one of the input targets T, or may be determined by the optimal variant search engine 70 in any manner.

As one example, optimal variant search engine 70 may iteratively and successively reduce the target performance metrics T until at least one program version satisfies the reduced metrics without exceeding the processor resources. That is, the optimal variant search engine 70 may reduce the performance metrics by some predetermined amount, determine whether any program version satisfies these reduced performance metrics and does not exceed the processor resources, and if not, continue to reduce the performance metrics, until at least one program version meets these criteria. The performance metrics are successively reduced until one program version satisfies the reduced metrics without exceeding the processor resources. If more than one program version meets these reduced criteria, the optimal variant search engine 70 selects one version in any manner, such as by selecting the highest performing version, the version that uses the least processor resources, or the like. If some of these program versions perform better in certain metrics while other versions perform better in other metrics, the optimal variant search engine 70 may select one program version according to any criteria. For example, selection may be performed by predetermining any hierarchy of metrics, with the program version that scores highest in the highest-priority metric being the version selected for execution. For instance, runtime may be selected as the priority, with the program version having lowest runtime therefore being the version selected. As another example, selection may be performed according to the program version that most exceeds any one or more metrics, exceeds the relaxed metrics by the greatest combined margin, according to user selection, or the like. Additionally, resource utilization recommender 40 may select the alternate program version that best meets some specified criteria, where the criteria may be any input criteria, such as known payload and target analysis methods, known resource selection or recommendation methods carried out by resource recommender 80, or based on any known statistical or other resource measure.

The optimal variant search engine 70 may handle this selected version in any manner, such as by simply initiating execution of the selected version, informing the user that an alternate (and likely lower-performing) version of the program must be executed, or invoking a known resource utilization recommender 40 to select the program version that best satisfies its specified criteria, where the criteria may be any input criteria, such as known payload and target analysis methods, known resource selection or recommendation methods, or based on any known statistical or other resource measure. In this manner, the optimal variant search engine 70 may select the program version that most closely satisfies the highest possible performance metrics.

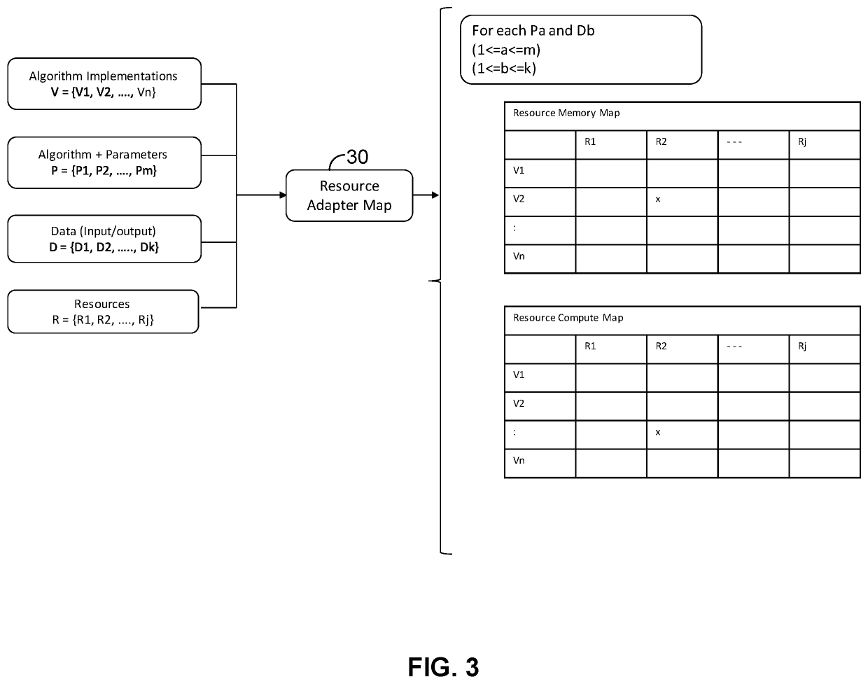

FIG. 3 conceptually illustrates operation of the system of FIGS. 1 and 2. As above, different versions V of a program are employed, and the algorithm and parameters P are input, along with the corresponding data payload D, and processor resources R. Runtime resource analyzer 10 then determines the processor resources used by each program version, and populates tables mapping the processor memory and compute resources used. For example, n different versions V of a program may be written, where each version V runs its algorithms or processes according to parameters P to process data at throughputs or payloads D. The available resources R, which are resources of a processor on which the versions V are to be run, are also input. As above, such resources R can include any compute resources including total available memory, total available processor resources as measured in, e.g., FLOPs, number of available processors, processor cores, warps, or other processing units such as GPUs or vGPUs, their capabilities, or the like.

Runtime resource analyzer 10 then determines the compute resources consumed by each program version V for each value of parameter P and each value of data throughput D. These compute resource values are then used to populate tables of memory and compute resources consumed. In particular, for each value of (P, D), runtime resource analyzer 10 generates a memory table and a compute table, as shown at the rightmost side of FIG. 3. The memory table entries are values of each peak processor memory resource R consumed by each program version V. That is, each program version Vn, run with specific values of parameters Pa and data throughput Db, consumes a certain amount of each processor memory resource Rj, and these amounts are populated in the appropriate fields of the memory resource table for Pa and Db. In similar manner, a compute resource table is also populated for Pa and Db. That is, each program version Vn, run with specific values of parameters Pa and data value Db, consumes a certain amount of each processor computational resource Rj, and these amounts are populated in the appropriate fields of the compute resource table for Pa and Db. This process may (in example, non-limiting embodiments) then be repeated for each different parameter value P and each different data value D to produce 2×a×b tables, or a pair of tables (memory and compute resource) for each Pa and each Db.

Program versions Vn may be any desired versions or variations of any process. For example, one program version may implement a deterministic model to solve a particular problem or application, another program version may instead implement one or more machine learning models such as neural network models to solve the same problem/application, and the like. These program versions Vn may be constructed to operate according to various parameters Pa. For instance, if the program versions Vn are medical image reconstruction programs, one parameter Pa may be the resolution at which the program versions Vn generate images, another parameter may be the number of images generated or the rate at which they are generated, and the like. Any parameters of any program type or application are contemplated. Program versions Vn are also written to operate according to various data volume or throughput values or value ranges Db. For example, medical image reconstruction programs may be written to handle data input of 200 MB, 500, MB, 1 GB, 2 GB, or any other values. For compute resource estimation purposes, data values D may instead represent ranges such as 200-500 MB, 1-2 GB, and the like.

As above, runtime resource analyzer 10 determines the compute resources consumed for each version Vn, each parameter Pa, and each data input setting Db, and populates memory and compute resource tables accordingly. Optimal variant search engine 70 may then compare entry values of these tables to the resources R of the processor, to select the default program version or an alternate program version that satisfies, or most closely satisfies, performance target T without exceeding the available resources R.

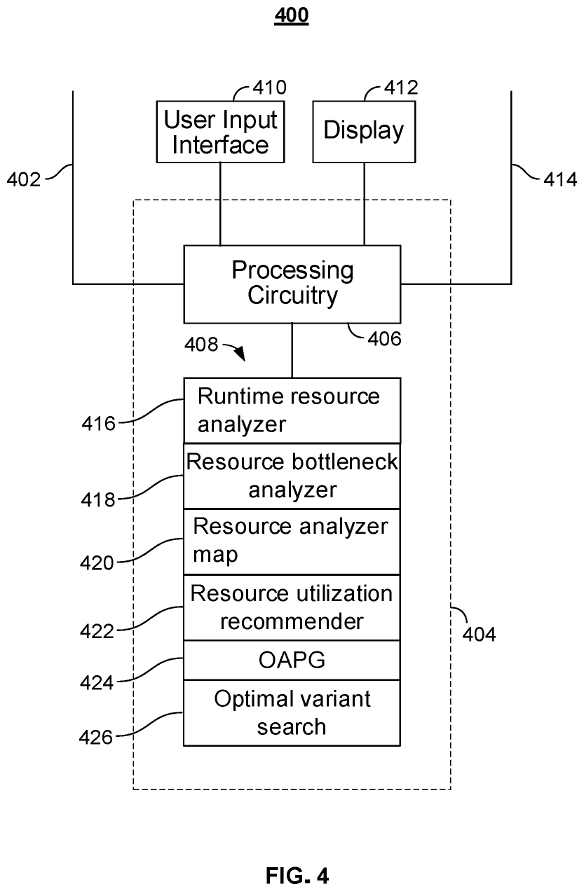

FIG. 4 is a generalized embodiment of an illustrative electronic computing device constructed for use according to embodiments of the disclosure. Here, computing device 400 may be any device capable of carrying out operations of embodiments of the disclosure. In particular, computing device 400 may execute the above described modules 10-50 to select a program version for execution, and may also execute the selected program version. That is, the various program versions as well as modules 10-50 may be packaged for loading onto and execution on any computing device 400, to select a program version most appropriate for execution on that particular device 400. In this manner, embodiments of the disclosure provide an adaptive set of programs that may be run on any processor of any device 400, with different program versions automatically selected according to the capabilities and resources of the specific device 400 on which it is to be executed.

As a nonlimiting example, computing device 400 may be a system on chip (SoC), embedded processor or microprocessor, or the like. Computing device 400 may transmit and receive data via input/output (hereinafter “I/O”) paths 402 and 414, which may be in electronic communication with any other device, e.g., through an electronic communications medium such as the public Internet. I/O path 402 may provide data (e.g., image data from camera 310 or the like) and other input to control circuitry 404, which includes processing circuitry 406 and storage 408. Control circuitry 404 may be used to send and receive commands, requests, and other suitable data using I/O path 402. I/O path 402 may connect control circuitry 404 (and specifically processing circuitry 406) to one or more communications paths. I/O functions may be provided by one or more of these communications paths but are shown as a single path in FIG. 4 to avoid overcomplicating the drawing.

Control circuitry 404 may be based on any suitable processing circuitry such as processing circuitry 406. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores). In some embodiments, processing circuitry may be distributed across multiple separate processors or processing units, for example, multiple of the same type of processing units (e.g., multiple NVIDIA® Tegra™ or Volta™ processors, Intel® Core™ processors, or the like) or multiple different processors (e.g., an Intel® Nervana™ processor and an NVIDIA® Volta™ processor, etc.). Any type and structure of processing circuitry may be employed. For example, processing circuitry 406 may include a multi-core processor, a multi-core processor structured as a graphics or computation pipeline for carrying out operations in parallel, a neuromorphic processor, any other parallel processor or graphics processor, or the like. In at least one embodiment, processing circuitry 406 may include, without limitation, a complex instruction set computer (“CISC”) microprocessor, a reduced instruction set computing (“RISC”) microprocessor, a very long instruction word (“VLIW”) microprocessor, a processor implementing a combination of instruction sets, or any other processor device, such as a digital signal processor or graphics processor, for example.

In some embodiments, control circuitry 404 executes instructions for determining gaze direction from input images, where these instructions may be embedded instructions or may be part of an application program running on an operating system. In at least one embodiment, computing device 100 may execute a version of the WINDOWS operating system available from Microsoft Corporation of Redmond, Wash., although other operating systems (UNIX and Linux for example), embedded software, and/or graphical user interfaces may also be used.

Memory may be an electronic storage device provided as storage 408 that is part of control circuitry 404. As referred to herein, the phrase “electronic storage device” or “storage device” should be understood to mean any device for storing electronic data, computer software, or firmware, such as random-access memory, read-only memory, hard drives, solid state devices, quantum storage devices, or any other suitable fixed or removable storage devices, and/or any combination of the same. Storage 408 may be used to store code modules as described below. Nonvolatile memory may also be used (e.g., to launch a boot-up routine and other instructions). Cloud-based storage may be used to supplement storage 408 or instead of storage 408.

Storage 408 may also store instructions or code for the above described program version selection processes, to carry out the operations of embodiments of the disclosure. In operation, processing circuitry 406 may retrieve and execute the instructions stored in storage 408, to carry out the processes herein.

Storage 408 is a memory that stores a number of programs or instruction modules for execution by processing circuitry 406. In particular, storage 408 may store a runtime resource analyzer module 416, a resource bottleneck analyzer module 418, a resource analyzer map module 420, a resource utilization recommender module 422, an OAPG module 424, and an optimal variant search module 426. Runtime resource analyzer module 416 may be a set of instructions for implementing runtime resource analyzer 10, resource bottleneck analyzer module 418 may be a set of instructions for implementing resource bottleneck analyzer 20, and resource analyzer map module 420 may be a set of instructions and data structures for implementing resource analyzer map 30. Likewise, resource utilization recommender module 422 may be a set of instructions for implementing resource utilization recommender 40, OAPG module 424 may be a set of instructions for implementing OAPG 50, and optimal variant search module 426 may be a set of instructions for implementing optimal variant search engine 70.

The computing device 400 may be standalone computing device such as a desktop or laptop computer, a server computer, or the like. However, embodiments of the disclosure are not limited to this configuration, and contemplate other implementations of computing device 400. For example, computing device 400 may be a remote computing device in wired or wireless communication with another electronic computing device via an electronic communications network such as the public Internet. In such latter embodiments, a user may remotely instruct computing device 400 to implement the processes described herein, to select program versions for execution on device 400.

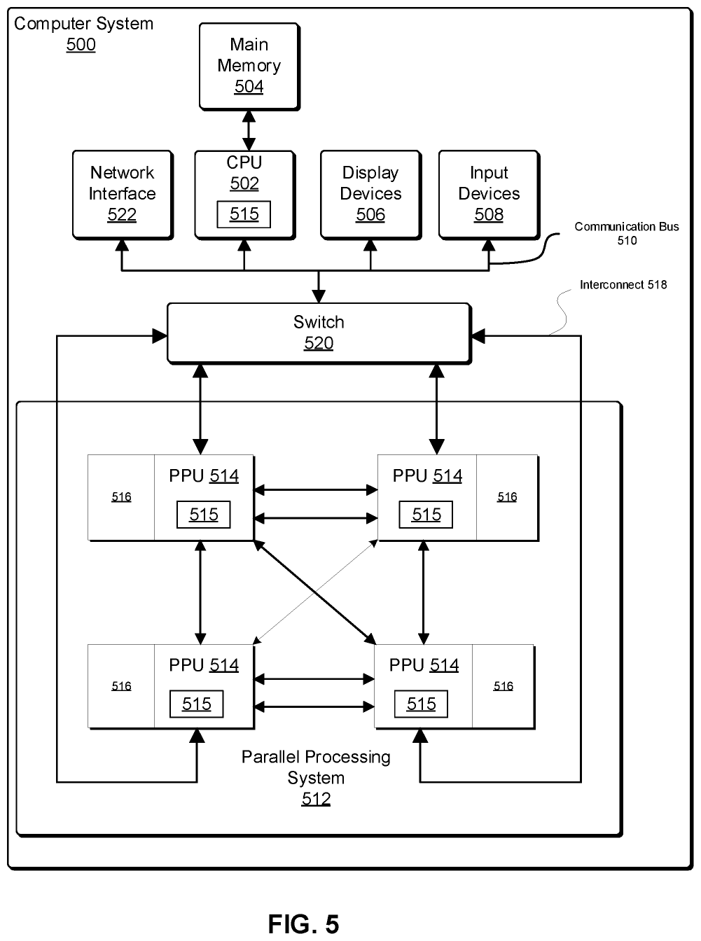

Computing device 400 may be any electronic computing device capable of selecting from among program versions for execution, and executing selected versions. For example, computing device 400 may be an embedded processor, a microcontroller, a local or remotely located desktop computer, tablet computer, or server. Furthermore, the computing device 400 may have any configuration or architecture that allows it to select and execute program versions according to embodiments of the disclosure. FIG. 5 illustrates one such configuration, in which computing device 400 is shown as a computer system 500 that is constructed with a parallel processing architecture for parallel processing of selected program versions. The computer system 500 of FIG. 5 may be employed, for example, in embodiments of the disclosure that use methods and processes for selecting programs and executing them. In at least one embodiment, computer system 500 comprises, without limitation, at least one central processing unit (“CPU”) 502 that is connected to a communication bus 510 implemented using any suitable protocol, such as PCI (“Peripheral Component Interconnect”), peripheral component interconnect express (“PCI-Express”), AGP (“Accelerated Graphics Port”), HyperTransport, or any other bus or point-to-point communication protocol(s). In at least one embodiment, computer system 500 includes, without limitation, a main memory 504 which may be any storage device, and control circuitry or logic (e.g., implemented as hardware, software, or a combination thereof). Data are stored in main memory 504 which may take the form of random access memory (“RAM”). In at least one embodiment, a network interface subsystem (“network interface”) 522 provides an interface to other computing devices and networks for receiving data from and transmitting data to other systems from computer system 500. Logic 515 is used to perform computational operations associated with one or more embodiments, and may be any processing circuitry. In particular, logic 515 may include, without limitation, code and/or data storage to store input/output data, and/or other parameters for carrying out any computational operations. Logic 515 may also include or be coupled to code and/or data storage to store code or other software to control timing and/or order of operations. Logic 515 may further include integer and/or floating point units (collectively, arithmetic logic units or ALUs) for carrying out operations on retrieved data as specified by stored code. In at least one embodiment, any portion of code and/or data storage may be included with other on-chip or off-chip data storage, including a processor's L1, L2, or L3 cache or system memory.

In at least one embodiment, parallel processing system 512 includes, without limitation, a plurality of parallel processing units (“PPUs”) 514 and associated memories 516. These PPUs 514 may be connected to a host processor or other peripheral devices via an interconnect 518 and a switch 520 or multiplexer. In at least one embodiment, parallel processing system 512 distributes computational tasks across PPUs 514 which can be parallelizable—for example, as part of distribution of computational tasks across multiple graphics processing unit (“GPU”) thread blocks. Memory may be shared and accessible (e.g., for read and/or write access) across some or all of PPUs 514, although such shared memory may incur performance penalties relative to use of local memory and registers resident to a PPU 214. In at least one embodiment, operation of PPUs 514 is synchronized through use of a command such as _syncthreads( ), wherein all threads in a block (e.g., executed across multiple PPUs 514) are to reach a certain point of execution of code before proceeding.

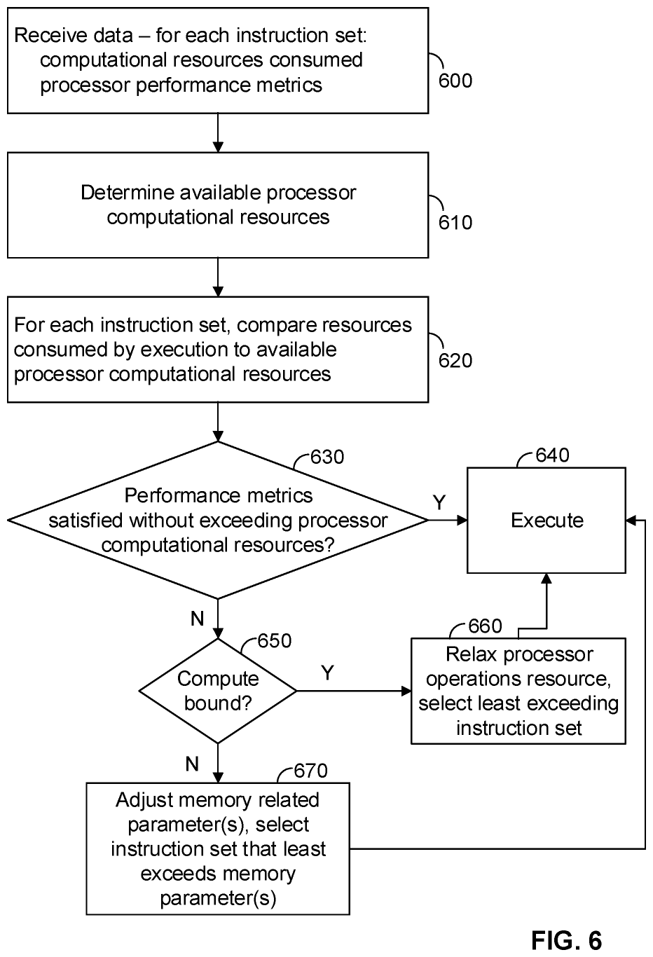

FIG. 6 is a flowchart illustrating process steps for improving processor resource utilization according to embodiments of the disclosure. As above, a number of program versions are stored in memory 408 of computing device 400, along with analysis modules 416-424 for carrying out the program selection and execution processes of embodiments of the disclosure. Runtime resource analyzer 416 then determines the computational resources, i.e., the maximum computational and memory resources, of processor 500 consumed by operation of each program version for each parameter P and data amount D, and receives the target T performance metrics the programs should meet when executed on the processor 500 (Step 600). As above, the runtime resource analyzer 416 populates processor memory and computational resource tables for each P and D.

The available resources of processor 500 are then received (Step 610). Resource bottleneck analyzer 418 compares the operational and memory resources consumed by each program version for each P and D, to the available computational resources of processor 500 (Step 620), by consulting the populated processor resource tables for each P and D. If a program version meets the target T performance metrics without exceeding the available processor compute and memory resources (Step 630), this program version is forwarded to the OAPG 424 for execution (Step 640). That is, if the table entries for the default program version and any P and D values do not exceed the corresponding computational resource values R of the processor 500, then the default version of the program is selected for execution by processor 500.

If, however, the default version exceeds one or more computational resources R of the processor 500 on which it is to be run, the resource bottleneck analyzer 418 determines whether the default program version is compute bound (Step 650), i.e., whether the table entries for the default program version exceed one or more of the available operational resources of processor 500. If so, the resource bottleneck analyzer 418 scans the populated tables to find an alternate program version that does not exceed the available computational resources of processor 500 while still meeting performance targets T (Step 660). If one such program version is found, it is forwarded to OAPG 424 for execution at Step 640. If more than one such program version is found, one of these versions is selected as above, such as by selection of the version with greatest or least processor utilization, selection by resource utilization recommended 40, or the like. If no program version falls within the available computational resources of processor 500 while still meeting performance targets T, the resource bottleneck analyzer 418 may take any action, such as issuing an error message, selecting a program version that least exceeds the available operational resources of processor 500, or relaxing or reducing the performance targets T until one program version satisfies the available computational resources of processor 500. The selected version may be executed on processor 500, or the user may be informed, e.g., that his or her requirements are not met but one version meets a reduced performance target.

If the default program version is instead memory bound, i.e., the table entries for the default program version exceed one or more of the available memory-related resources of processor 500, the resource bottleneck analyzer 418 scans the populated tables to find an alternate program version that does not exceed the available memory resources of processor 500 while still meeting performance targets T (Step 670). If one such program version is found, it is forwarded to OAPG 424 for execution at Step 640. If more than one such program version is found, one of these versions is selected as above, such as by selection of the version with greatest or least processor utilization, selection by resource utilization recommended 40, or the like. If no program version falls within the available memory resources of processor 500 while still meeting performance targets T, the resource bottleneck analyzer 418 may take any action, such as issuing an error message, selecting a program version that least exceeds the available memory resources of processor 500, or relaxing or reducing the performance targets T until one program version satisfies the available memory resources of processor 500, or the user may be informed, e.g., that his or her requirements are unmet but one version meets a reduced performance target.

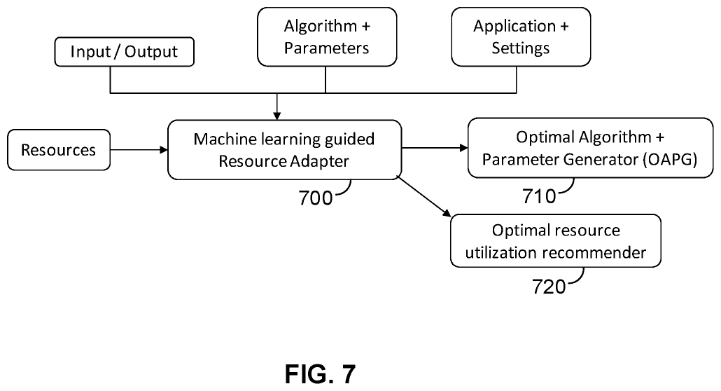

Program version selection may also be performed in any other manner or process. As one example, FIG. 7 is a block diagram conceptually illustrating operation of a system for improving processor resource utilization according to further embodiments of the disclosure. Here a machine learning guided resource adapter 700 takes the place of runtime resource analyzer 10, resource bottleneck analyzer 20, and resource adapter map 30. More specifically, adapter 700 takes the same inputs as adapter 10, and selects a program version that does not exceed the available memory resources of processor 500 while still meeting performance targets T. If multiple program versions satisfy these criteria, adapter 700 may employ one or more machine learning models trained to select the best or most optimal of these versions, given the input target T, parameters P, data input size/rate D, and available processor resources. Such machine learning models may be any models capable of selecting optimal or most desired items such as program variations, according to values of variables (e.g., T, P, D, etc.). For example, the machine learning models may be known classifiers or regression models trained to identify certain values or ranges of such variables as suitable for execution, and to select most optimal program variations accordingly.

In some embodiments, machine learning guided resource adapter 700 may select a program variation that best falls within the computational resources of processor 500 while still meeting performance targets T, and may pass this variation to OAPG 710 for execution on processor 500. If multiple program variations meet these criteria, optimal resource utilization recommender 720 may select the optimal such variation for execution, according to any suitable criteria, such as via the machine learning model of adapter 700, a known analysis of which variation most optimally utilizes the resources of processor 500, or the like. Selection of program variations may include relaxing performance targets T as above if desired.

The foregoing description, for purposes of explanation, used specific nomenclature to provide a thorough understanding of the disclosure. However, it will be apparent to one skilled in the art that the specific details are not required to practice the methods and systems of the disclosure. Thus, the foregoing descriptions of specific embodiments of the present invention are presented for purposes of illustration and description. They are not intended to be exhaustive or to limit the invention to the precise forms disclosed. Many modifications and variations are possible in view of the above teachings. For example, program variations can be any implementations of any process or method, using any parameters and operating on any type, rate, or amount of data input. Program variations can be compared to any computational resources of any processor. Methods and processes of embodiments of the disclosure may also be employed to select programs for execution on any type of processor, whether GPU, CPU, or the like. The embodiments were chosen and described in order to best explain the principles of the invention and its practical applications, to thereby enable others skilled in the art to best utilize the methods and systems of the disclosure and various embodiments with various modifications as are suited to the particular use contemplated. Additionally, different features of the various embodiments, disclosed or otherwise, can be mixed and matched or otherwise combined so as to create further embodiments contemplated by the disclosure.

What is claimed is:

1. A method of improving utilization of a graphics processing unit (GPU), the method comprising:
receiving, using processing circuitry, data corresponding to computational resources consumed by execution of a plurality of sets of instructions for execution by a GPU, and one or more performance metrics for the GPU;
determining available computational resources of the GPU;
using the data, comparing the computational resources consumed by execution of the sets of instructions to the available computational resources of the GPU, to select one of the sets of instructions that, when executed by the GPU, satisfies the one or more performance metrics for the GPU without exceeding the computational resources of the GPU; and
executing the selected set of instructions using the GPU.

2. The method of claim 1, further comprising, if no set of instructions, when executed by the GPU, satisfies the one or more performance metrics for the GPU without exceeding the computational resources of the GPU:
selecting one or more reduced performance metrics for the GPU; and
using the data, selecting the set of instructions that, when executed by the GPU, satisfies the one or more reduced performance metrics for the GPU without exceeding the computational resources of the GPU.

3. The method of claim 2, further comprising, if no set of instructions, when executed by the GPU, satisfies the one or more reduced performance metrics for the GPU without exceeding the computational resources of the GPU:
using the data, selecting the set of instructions that, executed by the GPU, most closely satisfies the one or more reduced performance metrics for the GPU without exceeding the computational resources of the GPU.

4. The method of claim 2, wherein the one or more performance metrics comprise one or more of an end-to-end runtime or a minimum rate at which information is generated, and wherein the one or more reduced performance metrics comprise one or more of an increased end-to-end runtime or a reduced minimum rate at which information is generated.

5. The method of claim 1, wherein the computational resources and the available computational resources each comprise one or more of:
amounts of GPU memory consumed by execution of the respective sets of instructions,
floating point operations per second performed in execution of the respective sets of instructions, or
total compute operations performed in execution of the respective sets of instructions.

6. The method of claim 1, wherein the computational resources further comprise a memory resource of the GPU, and wherein the comparing further comprises:
using the data, selecting one of the sets of instructions that, when executed by the GPU, does not exceed the memory resource of the GPU.

7. The method of claim 6, further comprising, if every set of instructions, when executed by the GPU, exceeds the memory resource of the GPU, selecting one of the sets of instructions that least exceeds the memory resource of the GPU.

8. The method of claim 1, wherein the computational resources further comprise a processor operations resource of the GPU, and wherein the comparing further comprises:
using the data, selecting one of the sets of instructions that, when executed by the GPU, does not exceed the processor operations resource of the GPU.

9. The method of claim 8, further comprising, if every set of instructions, when executed by the GPU, exceeds the processor operations resource of the GPU, selecting one of the sets of instructions that least exceeds the processor operations resource of the GPU.

10. The method of claim 1, wherein the sets of instructions each comprise instructions for medical image reconstruction.

11. The method of claim 1, wherein each of the sets of instructions is configured to perform the same one or more computational processes.

12. The method of claim 1, wherein at least one of the sets of instructions is a set of instructions for implementing one or more neural network models.

13. The method of claim 1, wherein the GPU comprises a virtual GPU (vGPU).

14. The method of claim 13, further comprising selecting a number of vGPUs available for execution of the sets of instructions.

15. A non-transitory computer readable medium having data encoded thereon and instructions included thereon for execution by processing circuitry, the data and instructions comprising:
first sets of instructions for execution by a graphics processing unit (GPU); and
data corresponding to computational resources consumed by execution of the first sets of instructions by the GPU, and one or more performance metrics for the GPU; and
a second set of instructions for execution by processing circuitry to:
determine available computational resources of the GPU;
using the data, compare the computational resources consumed by execution of the first sets of instructions to the available computational resources of the GPU, to select one of the sets of instructions that, when executed by the GPU, satisfies the one or more performance metrics for the GPU without exceeding the computational resources of the GPU; and
initiate execution of the selected set of instructions by the GPU.

16. The non-transitory computer readable medium of claim 15, wherein the second set of instructions further comprises instructions to, if no first set of instructions, when executed by the GPU, satisfies the one or more performance metrics for the GPU without exceeding the computational resources of the GPU:
select one or more reduced performance metrics for the GPU; and
using the data, select the set of instructions that, when executed by the GPU, satisfies the one or more reduced performance metrics for the GPU without exceeding the computational resources of the GPU.

17. The non-transitory computer readable medium of claim 16, wherein the second set of instructions further comprises instructions to, if no first set of instructions, when executed by the GPU, satisfies the one or more reduced performance metrics for the GPU without exceeding the computational resources of the GPU:
using the data, select the set of instructions that, executed by the GPU, most closely satisfies the one or more reduced performance metrics for the GPU without exceeding the computational resources of the GPU.

18. The non-transitory computer readable medium of claim 16, wherein the one or more performance metrics comprise one or more of an end-to-end runtime or a minimum rate at which information is generated, and wherein the one or more reduced performance metrics comprise one or more of an increased end-to-end runtime or a reduced minimum rate at which information is generated.

19. The non-transitory computer readable medium of claim 15, wherein the computational resources and the available computational resources each comprise one or more of:
amounts of GPU memory consumed by execution of the respective sets of instructions,
floating point operations per second performed in execution of the respective sets of instructions, or
total compute operations performed in execution of the respective sets of instructions.

20. The non-transitory computer readable medium of claim 15, wherein the computational resources further comprise a memory resource of the GPU, and wherein the comparing further comprises:
using the data, selecting one of the sets of instructions that, when executed by the GPU, does not exceed the memory resource of the GPU.

21. The non-transitory computer readable medium of claim 20, wherein the second set of instructions further comprises instructions to, if every first set of instructions, when executed by the GPU, exceeds the memory resource of the GPU, select one of the sets of instructions that least exceeds the memory resource of the GPU.

22. The non-transitory computer readable medium of claim 15, wherein the computational resources further comprise a processor operations resource of the GPU, and wherein the comparing further comprises:
using the data, selecting one of the sets of instructions that, when executed by the GPU, does not exceed the processor operations resource of the GPU.

23. The non-transitory computer readable medium of claim 22, wherein the second set of instructions further comprises instructions to, if every first set of instructions, when executed by the GPU, exceeds the processor operations resource of the GPU, select one of the sets of instructions that least exceeds the processor operations resource of the GPU.

24. The non-transitory computer readable medium of claim 15, wherein the first sets of instructions each comprise instructions for medical image reconstruction.

25. The non-transitory computer readable medium of claim 15, wherein each of the first sets of instructions is configured to perform the same one or more computational processes.

26. The non-transitory computer readable medium of claim 15, wherein at least one of the first sets of instructions is a set of instructions for implementing one or more neural network models.

27. The -transitory computer readable medium of claim 15, wherein the GPU comprises a virtual GPU (vGPU).

28. The -transitory computer readable medium of claim 27, wherein the second set of instructions further comprises instructions to select a number of vGPUs available for execution of the sets of instructions.
